(19) United States
(12) Patent Application Publication
(10) Pub. No.: US 2025/0332388 A1
(43) Pub. Date: Oct. 30, 2025

(54) GUIDEWIRE SYSTEMS AND METHODS OF USE

(71) Applicant: Covidien LP, Mansfield, MA (US)

(72) Inventors: Ashok R. Nageswaran, Irvine, CA (US); Daniel Deen, Long Beach, CA (US)

(21) Appl. No.: 19/189,488

(22) Filed: May 13, 2025

Related U.S. Application Data
(60) Provisional application No. 63/639,231, filed on Apr. 26, 2024.

(51) Int. Cl. A61M 25/09; A61B 17/22; A61B 17/221

(52) U.S. Cl. CPC A61M 25/09041; A61B 17/221; A61B 2017/22038; A61B 2017/22079; A61B 2017/2212

(57) ABSTRACT

Guidewire systems and associated methods of use are disclosed herein. According to some embodiments, the guidewire system includes a guidewire comprising a tubular sidewall defining a lumen and an elongate delivery member slidably positioned within the lumen. The guidewire system can further include an interventional element disposed at a distal region of the delivery member and positioned within the lumen of the guidewire in a collapsed configuration. The delivery member is configured to be pushed distally within the guidewire lumen to expel the interventional element from the lumen, thereby allowing the interventional element to expand to an expanded configuration.

### CROSS-REFERENCE TO RELATED APPLICATIONS

This application claims the benefit of and priority to U.S. Provisional Patent Application Ser. No. 63/639,231 filed Apr. 26, 2024, the entire disclosure of which is incorporated by reference herein.

### TECHNICAL FIELD

The present technology relates to guidewire systems and methods of use.

### BACKGROUND

In the field of interventional cardiology and vascular surgery, accessing and navigating through intricate networks of blood vessels is a fundamental aspect of various diagnostic and therapeutic procedures. Guidewires play a pivotal role in these procedures, serving as essential tools for clinicians to reach remote or challenging anatomical sites with precision and safety. Traditionally, guidewires have been constructed from materials such as stainless steel or nitinol, featuring a tapered or flexible design to facilitate navigation through tortuous vasculature. However, existing guidewire technologies often face limitations when encountering highly stenosed or tortuous vessels, which can impede effective advancement and compromise procedural outcomes. Furthermore, the risk of vessel trauma or perforation remains a significant concern, particularly in cases where the guidewire encounters resistance or requires manipulation in delicate vascular territories. Finally, exchanging multiple other devices over the guidewire and/or exchanging the guidewire for an interventional device can be time-consuming for the physician. As such, there exists a need within the medical community for an improved guidewire design capable of enhancing navigational capabilities, minimizing procedural time, and accommodating a broader range of anatomical challenges.

### SUMMARY

The subject technology is illustrated, for example, according to various aspects described below, including with reference to FIGS. 1 and 2. Various examples of aspects of the subject technology are described as numbered examples (1, 2, 3, etc.) for convenience. These are provided as examples and do not limit the subject technology.

Example 1. A guidewire system for traversing a patient's vasculature, the guidewire system comprising a guidewire comprising a tubular sidewall defining a lumen therein, the guidewire extending between a proximal portion and a distal portion, and wherein the distal portion of the guidewire is configured to be intravascularly delivered to a treatment site within a blood vessel; an elongate delivery member slidably positioned within the lumen of the guidewire, the delivery member having a proximal region and a distal region; and an interventional element carried by the distal region of the delivery member and configured to be positioned within the lumen of the guidewire in a collapsed configuration, wherein the delivery member is configured to be pushed distally within the lumen to expel the interventional element from the lumen, thereby allowing the interventional element to expand to an expanded configuration.

Example 2: The guidewire system of Example 1, wherein the guidewire has an outer diameter of 0.024 inches or less.

Example 3: The guidewire system of Example 1 or Example 2, further comprising a stiffening member fixed to the distal portion of the delivery member, proximal of the interventional element.

Example 4: The guidewire system of Example 3, wherein the stiffening member comprises a coil.

Example 5: The guidewire system of any one of Examples 1 to 3, further comprising a visualization member fixed to the distal portion of the delivery member, distal of the interventional element.

Example 6: The guidewire system of Example 5, wherein the visualization member comprises a coil.

Example 7: The guidewire system of Example 1, further comprising a stiffening member fixed to the distal portion of the delivery member, proximal of the interventional element; and a visualization member fixed to the distal portion of the delivery member, distal of the interventional element.

Example 8: The guidewire system of Example 7, wherein one or both of the stiffening member and the visualization member comprises a coil.

Example 9: The guidewire system of any one of Examples 1 to 8, wherein at least a distal portion of the sidewall of the guidewire comprises a laser-cut hypotube.

Example 10: The guidewire system of any one of Examples 1 to 9, wherein the distal region of the delivery member has an outer diameter than is smaller than an outer diameter of the proximal region.

Example 11: The guidewire system of any one of Examples 1 to 10, wherein the interventional element is an expandable stent comprising a plurality of interconnected struts.

Example 12: The guidewire system of any one of Examples 1 to 11, wherein at least a distal portion of the sidewall of the guidewire includes a plurality of slits.

Example 13: A treatment system, comprising: a guidewire system for traversing a patient's vasculature, the guidewire system comprising: a guidewire comprising a tubular sidewall defining a lumen therein, the guidewire extending between a proximal portion and a distal portion, and wherein the distal portion of the guidewire is configured to be intravascularly delivered to a treatment site within a blood vessel; an elongate delivery member slidably positioned within the lumen of the guidewire, the delivery member having a proximal region and a distal region; and an interventional element carried by the distal region of the delivery member and configured to be positioned within the lumen of the guidewire in a collapsed configuration, wherein the delivery member is configured to be pushed distally within the lumen to expel the interventional element from the lumen, thereby allowing the interventional element to expand to an expanded configuration; and a catheter defining a lumen, wherein the guidewire system is configured to be delivered through the lumen of the catheter to the treatment site.

Example 14: The treatment system of Example 13, wherein the catheter is a guide catheter.

Example 15: The treatment system of Example 13, wherein the catheter is an aspiration catheter.

Example 16: The treatment system of any one of Examples 13 to 15, wherein the treatment system does not include a microcatheter.

Example 17: A method, comprising: advancing a distal portion of a guidewire system to a treatment site within a blood vessel, the guidewire system comprising a guidewire defining a lumen, an elongate delivery member slidably positioned within the lumen, and an interventional element carried by the distal region of the delivery member and positioned within the lumen in a radially constrained configuration; delivering a catheter over the guidewire system to the treatment site; and expelling the interventional element from the lumen of the guidewire, thereby allowing the interventional element to expand into an expanded configuration.

Example 18: The method of Example 17, wherein the guidewire system is not delivered over another guidewire.

Example 19: The method of Example 17 or Example 18, further comprising engaging clot material with the interventional element.

Example 20: The method of any one of Examples 17 to 19, wherein the catheter is an aspiration catheter.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a side view of a guidewire system configured in accordance with several embodiments of the present technology.

FIG. 2 is a side view of an example interventional element for use with the guidewire systems of the present technology.

### DETAILED DESCRIPTION

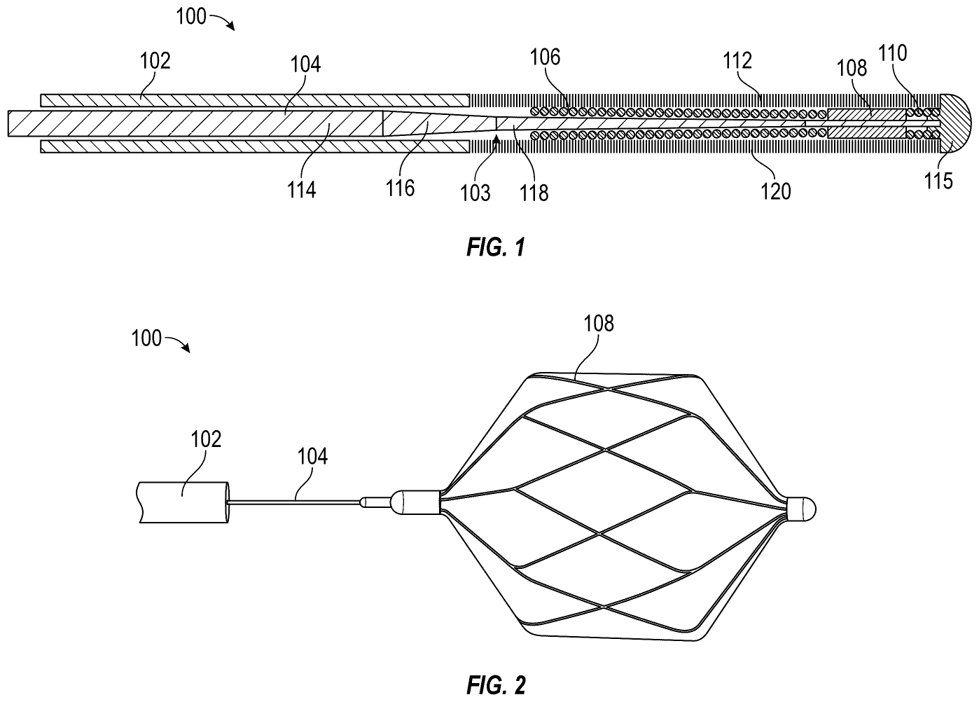

Acute ischemic stroke procedures typically require a guidewire, aspiration catheter, microcatheter and stent to retrieve clot material. Initially, the guidewire is used to access the target vessel. Next, an aspiration catheter is tracked over the guidewire to reach the target vessel, and a microcatheter is then tracked over the guidewire, through the aspiration catheter, to reach the target vessel. The guidewire is then removed from the patient and a stent is tracked through the microcatheter. The numerous devices used to reach the target vessel are time-consuming. The guidewire systems of the present technology address this issue. Disclosed herein are tubular guidewires configured to contain an interventional element, such as a stent, with a relatively low radial outward force. The present technology thus eliminates the need for a microcatheter, thereby simplifying access and reducing procedure time. Specific details of several embodiments of the technology are described below with reference to FIGS. 1 and 2.

FIG. 1 shows a guidewire system 100 for traversing a patient's vasculature configured in accordance with the present technology. The guidewire system 100 can comprise a guidewire 102, an elongate delivery member 104, and an interventional element 108. Each of the guidewire 102 and elongate delivery member 104 can have corresponding proximal portions configured to be positioned at an extracorporeal location for manipulation by a user, and corresponding distal portions configured to be positioned within a blood vessel (such as a cerebral blood vessel) of a patient. The guidewire 102 can comprise a tubular sidewall defining a lumen 103, and the delivery member 104 can be configured to be slidably disposed within the lumen 103. The interventional element 108 can be disposed at the distal portion of the delivery member 104 and is configured to be positioned within the lumen 103 of the guidewire 102 in a collapsed and/or radially constrained configuration (as shown schematically in FIG. 1). The delivery member 104 is configured to be pushed distally within the lumen 103 (or the guidewire 102 pulled proximally relative to the delivery member 104) to expel the interventional clement 108 from the lumen 103, thereby allowing the interventional element 108 to expand to an expanded configuration.

In general, the guidewire system 100 is flexible enough to traverse a circuitous path through the vascular system, and yet have sufficient pushability to transmit a pushing force from a remote proximal portion of the guidewire system 100, along a winding path, to the distal portion of the guidewire system 100. The guidewire system 100 also has sufficient torsional stiffness to reliably transmit rotational force applied at the proximal portion to the distal portion so that the guidewire system can be steered through the branches of vessels of the vascular system. The guidewire sidewall can have an outer diameter of 0.024 inches or less, and in some cases 0.021 inches or less. As discussed in greater detail below, an aspiration and/or guide catheter can be delivered over the guidewire system 100 (e.g., the guidewire system 100 is configured to be slidably disposed within an aspiration and/or guide catheter lumen).

In some embodiments, the sidewall of the guidewire 102 can be configured to have a flexible region 112 that is more flexible than the more proximal regions of the sidewall. In some cases, the flexible region 112 comprises a plurality of slits 120. For example, the flexible region 112 can comprise a laser-cut hypotube. In some examples, the flexible region 112 comprises a tubular coil.

As shown in FIG. 1, the delivery member 104 can have a varying diameter. For example, the delivery member can have a first region 114 having a first substantially constant or distally tapering diameter, a second region 116 distal of the first region 114 have a distally tapering diameter, and a third region 118 having a third substantially constant or distally tapering diameter. In some embodiments, the flexible region 112 of the guidewire 102 is aligned with all or some of the third region 118.

The guidewire system 100 can further comprise a stiffening member 106 coupled to the distal portion of the delivery member 104, proximal of the interventional element 108 along a longitudinal axis of the system 100. The stiffening member 106 can coincide with the smaller diameter third region 118 of the delivery member 102 and is configured to provide additional support to the delivery member 102. In some cases the stiffening member 106 comprises a tubular coil defining a lumen, and the delivery member 104 is disposed within the lumen. The stiffening member 106 can be fixed to the delivery member 104 such that the stiffening member 104 cannot move longitudinally or rotationally relative to the delivery member 104. A distal end of the stiffening member 106 can terminate proximal of a proximal end of the interventional element 108 such that the interventional element 108 and the stiffening member 106 do not longitudinally overlap. While the stiffening member 106 is shown as a coil in FIG. 1, in other embodiments the stiffening member 106 can have other forms, such as a radiopaque tube and/or band and others.

The guidewire system 100 can further comprise a visualization member 110 coupled to the distal portion of the delivery member 104, distal of the interventional element 108 along a longitudinal axis of the system 100. The visualization member 110 can include a radiopaque material and is configured to provide visualization of the distal end of the delivery member 104. In some cases the visualization member 110 comprises a tubular coil defining a lumen, and the delivery member 104 is disposed within the lumen. The visualization member 110 can be fixed to the delivery member 104 such that the visualization member 110 cannot move longitudinally or rotationally relative to the delivery member 104. A proximal end of the visualization member 110 can be disposed distal of a distal end of the interventional element 108 such that the interventional element 108 and the visualization member 110 do not longitudinally overlap. While the visualization member 110 is shown as a coil in FIG. 1, in other embodiments the visualization member 110 can have other forms, such as a radiopaque tube and/or band and others.

A length of the stiffening member 106 can be greater than a length of the visualization member 110. In other embodiments, the stiffening member 106 and visualization member 110 can have the same lengths.

In some embodiments, the guidewire system 100 does not include one or both of the stiffening member 106 and the visualization member 110.

The guidewire system 100 can optionally comprise a distal tip 115 disposed at the distal end of the delivery member 104. The distal tip 115 can comprise a rounded distal surface and have an outer diameter matching that of the guidewire 102. As such, the distal tip 115 closes an opening at the distal end of the guidewire 102 and provides an atraumatic surface for traversing the vasculature. In some embodiments the distal tip 115 comprises a radiopaque material. In some embodiments the guidewire system 100 does not include a distal tip 115.

FIG. 2 shows the guidewire system 100 with the interventional element 108 released from the guidewire lumen 103, in an expanded configuration. The interventional clement 108 can be configured to treat a vascular condition at a treatment site within the blood vessel. The interventional element 108 can be implantable (e.g., a flow diverter for implantation in the blood vessel lumen) or non-implantable (e.g., a stentriever for removal of clot material). As such, in some examples the interventional element 108 can be fixedly coupled to the distal portion of the delivery member 104, and in other examples the interventional element 108 is detachably coupled to the delivery member 104. As shown in FIG. 2, in some embodiments the interventional element 108 can comprise an expandable stent comprising a plurality of interconnected struts. It will be appreciated that other structures are possible. The interventional element can have a relatively low radial outward force.

In use, a distal portion of the guidewire 102, with the delivery member 104 and interventional element 108 contained therein, can be advanced to a treatment site within a blood vessel. The guidewire system 100 is advanced through the vasculature alone, at the beginning of the procedure, without guidance from another guidewire. Another catheter can optionally be delivered over the guidewire system 100, such as an aspiration catheter or delivery catheter. The interventional element can then be expelled from the lumen of the guidewire, thereby allowing the interventional element to expand into an expanded configuration for treatment at the treatment site (e.g., such as engagement and removal of clot material). Use of the guidewire to deliver and deplo the interventional element, rather than a separate microcatheter delivered over the guidewire, eliminates the need for a microcatheter, thereby simplifying access and reducing procedure time.

### Conclusion

Although many of the embodiments are described above with respect to systems, devices, and methods for treating ischemic stroke, the technology is applicable to other applications and/or other approaches, such as deployment of any implantable or non-implantable interventional device at a remote location within a blood vessel. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1 and 2.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A guidewire system for traversing a patient's vasculature, the guidewire system comprising:
a guidewire comprising a tubular sidewall defining a lumen therein, the guidewire extending between a proximal portion and a distal portion, and wherein the distal portion of the guidewire is configured to be intravascularly delivered to a treatment site within a blood vessel;
an elongate delivery member slidably positioned within the lumen of the guidewire, the delivery member having a proximal region and a distal region; and
an interventional element carried by the distal region of the delivery member and configured to be positioned within the lumen of the guidewire in a collapsed configuration,
wherein the delivery member is configured to be pushed distally within the lumen to expel the interventional element from the lumen, thereby allowing the interventional element to expand to an expanded configuration.

2. The guidewire system of claim 1, wherein the guidewire has an outer diameter of 0.024 inches or less.

3. The guidewire system of claim 1, further comprising a stiffening member fixed to the distal portion of the delivery member, proximal of the interventional element.

4. The guidewire system of claim 3, wherein the stiffening member comprises a coil.

5. The guidewire system of claim 1, further comprising a visualization member fixed to the distal portion of the delivery member, distal of the interventional element.

6. The guidewire system of claim 5, wherein the visualization member comprises a coil.

7. The guidewire system of claim 1, further comprising:
a stiffening member fixed to the distal portion of the delivery member, proximal of the interventional element; and
a visualization member fixed to the distal portion of the delivery member, distal of the interventional element.

8. The guidewire system of claim 7, wherein one or both of the stiffening member and the visualization member comprise a coil.

9. The guidewire system of claim 1, wherein at least a distal portion of the sidewall of the guidewire comprises a laser-cut hypotube.

10. The guidewire system of claim 1, wherein the distal region of the delivery member has an outer diameter than is smaller than an outer diameter of the proximal region.

11. The guidewire system of claim 1, wherein the interventional element is an expandable stent comprising a plurality of interconnected struts.

12. The guidewire system of claim 1, wherein at least a distal portion of the sidewall of the guidewire includes a plurality of slits.

13. A treatment system, comprising:
a guidewire system for traversing a patient's vasculature, the guidewire system comprising:
a guidewire comprising a tubular sidewall defining a lumen therein, the guidewire extending between a proximal portion and a distal portion, and wherein the distal portion of the guidewire is configured to be intravascularly delivered to a treatment site within a blood vessel;
an elongate delivery member slidably positioned within the lumen of the guidewire, the delivery member having a proximal region and a distal region; and
an interventional element carried by the distal region of the delivery member and configured to be positioned within the lumen of the guidewire in a collapsed configuration, wherein the interventional element is configured to be expelled from the guidewire lumen, thereby allowing the interventional element to expand to an expanded configuration; and
a catheter defining a lumen, wherein the guidewire system is configured to be delivered through the lumen of the catheter to the treatment site.

14. The treatment system of claim 13, wherein the catheter is a guide catheter.

15. The treatment system of claim 13, wherein the catheter is an aspiration catheter.

16. The treatment system of claim 13, wherein the treatment system does not include a microcatheter.

17. A method, comprising:
advancing a distal portion of a guidewire system to a treatment site within a blood vessel, the guidewire system comprising a guidewire defining a lumen, an elongate delivery member slidably positioned within the lumen, and an interventional element carried by the distal region of the delivery member and positioned within the lumen in a radially constrained configuration;
delivering a catheter over the guidewire system to the treatment site; and
expelling the interventional element from the lumen of the guidewire, thereby allowing the interventional element to expand into an expanded configuration.

18. The method of claim 17, wherein the guidewire system is not delivered over another guidewire.

19. The method of claim 17. further comprising engaging clot material with the interventional element.

20. The method of claim 17, wherein the catheter is an aspiration catheter.
